SEQUENCE LISTING (10 sequences)

SEQ ID NO: 1 - DNA, 19 bp, Artificial Sequence
  Forward Primer 515F
gtgccagcmgccgcggtaa

SEQ ID NO: 2 - DNA, 20 bp, Artificial Sequence
  Reverse Primer 806R
ggactachvgggtwtctaat

SEQ ID NO: 3 - DNA, 19 bp, Artificial Sequence
  Forward Primer gITS7
gtgartcatcgartctttg

SEQ ID NO: 4 - DNA, 20 bp, Artificial Sequence
  Reverse Primer ITS4
tcctccgcttattgatatgc

SEQ ID NO: 5 - DNA, 16 bp, Artificial Sequence
  Forward Primer 530F
gtgccagcmgccgcgg

SEQ ID NO: 6 - DNA, 16 bp, Artificial Sequence
  Reverse Primer 23SR
gggttbccccattcrg

SEQ ID NO: 7 - DNA, 19 bp, Artificial Sequence
  Forward Primer Achromobacter
tttaactaccgagctagag

SEQ ID NO: 8 - DNA, 20 bp, Artificial Sequence
  Forward Primer Opitutus
aactgcgtctgaaactacat

SEQ ID NO: 9 - DNA, 19 bp, Artificial Sequence
  Forward Primer Pseudomonas
aactgactgactagagtat

SEQ ID NO: 10 - DNA, 18 bp, Artificial Sequence
  Forward Primer Sulfuritalea
aactgcgtttgtgactgt

(19) United States
(12) Patent Application Publication
(10) Pub. No.: US 2020/0000099 A1
(43) Pub. Date: Jan. 2, 2020

(54) ORGANISMS ANTAGONISTIC TO XYLELLA FASTIDIOSA

(71) Applicant: The Regents of the University of California, Oakland, CA (US)

(72) Inventors: Philippe Rolshausen, Riverside, CA (US); Mary Caroline Roper, Riverside, CA (US); James Borneman, Riverside, CA (US)

(21) Appl. No.: 16/482,250

(22) PCT Filed: Jan. 30, 2018
(86) PCT No.: PCT/US18/16010

§ 371 Date: Jul. 30, 2019

Related U.S. Application Data
(60) Provisional application No. 62/452,870, filed on Jan. 31, 2017.

(51) Int. Cl. A01N 63/02; A01N 41/04; A01N 43/90; C12N 15/82

(52) U.S. Cl. CPC A01N 63/02; C12N 15/82; A01N 43/90; A01N 41/04

(57) ABSTRACT

The disclosure describes microorganisms and preparations useful for inhibiting infection by Xylella sp. and in the treatment of Pierce's Disease.

### CROSS REFERENCE TO RELATED APPLICATIONS

This claims priority from U.S. Provisional Application Ser. No. 62/452,870, filed Jan. 31, 2017, the disclosures of which are incorporated herein by reference.

### FIELD OF THE INVENTION

The invention relates generally to methods and compositions useful for treating plant diseases, in particular infections by pathogens.

### BACKGROUND

Xylella fastidiosa (Xf) is a xylem limited bacterium that dwells in a wide array of plant species and the foregut of certain xylem feeding insects. The insects serve to transport Xf from plant to plant when feeding. Xf does not harm its host insect and most plant hosts are not harmed by Xf. However certain plant species are susceptible to strains of Xf and disease can occur. Such diseases include grapevine Pierce's Disease (PD).

### SUMMARY

The disclosure provides a method for preventing infection and/or protecting a plant from infection by Xylella sp. microorganisms comprising inoculating the plant with (i) an anti-Xf endophytic microorganism selected from the group consisting of Pseudomonas fluorescens, Achromobacter xylosoxidans and Achromobacter sp., and optionally one or more of Cochliobolus sp., Aspergilllus sp., Phaeosphaeria sp., Ulocladium sp., Dicostroma sp., Geomyces sp. and Cryptococcus sp., and any combination thereof; and/or (ii) an extract comprising an anti-Xf agent that inhibits Xylella sp. infection obtained from any one or more of the microorganisms identified above. In one embodiment, the Xylella sp. is Xylella fastidiosa. In another embodiment, the anti-Xf endophytic microorganism is Pseudomonas fluorescens and/or Achromobacter xylosoxidans.

The disclosure provides a method for preventing infection and/or protecting a plant from infection by Xylella sp. microorganisms comprising inoculating the plant with (i) an anti-Xf endophytic microorganism selected from the group consisting of Pseudomonas sp. and/or Achromobacter sp. and/or (ii) an extract comprising an anti-Xf agent that inhibits Xylella sp. infection. In one embodiment, the Xylella sp. is Xylella fastidiosa. In another embodiment, the anti-Xf endophytic microorganism is Pseudomonas fluorescens and/or Achromobacter xylosoxidans. In still another embodiment, the plant is a monocotyleyledonous plant. In yet another embodiment, the plant is a dictotyledonous plant. In still another or further embodiment, the plant is selected from the group consisting of grape, oleander, oak, almond, peach, pear, citrus, coffee, maple, mulberry, elm, sycamore, and alfalfa. In another embodiment, the anti-Xf endophytic microorganism is inoculated in the xylem of the plant. In yet another embodiment, the anti-Xf agent comprises the general structural formula selected from the group consisting of:

### (a) Formula Ia:

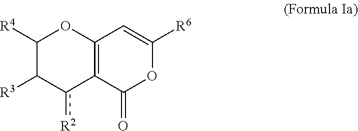

wherein, R² is selected from the group comprising halo, CN, O, OH, NH, NH₂, S, SH, and CH₂; R³-R⁴ are each individually selected from the group comprising H, halo, hydroxyl, cyano, thiol, mino, optionally substituted (C₁-C₆)alkyl, and optionally substituted (C₁-C₆)alkenyl; and R⁶ is selected from the group comprising H, optionally substituted (C₁-C₆)alkyl, and optionally substituted (C₁-C₆)alkenyl, and optionally substituted heterocycle;

### (b) Formula 1b:

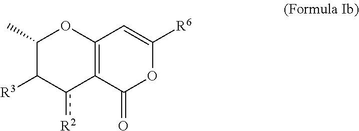

wherein, R² is selected from the group comprising halo, CN, O, OH, NH, NH₂, S, SH, and CH₂; R³ is individually selected from the group comprising H, halo, hydroxyl, cyano, thiol, amino, optionally substituted (C₁-C₆)alkyl, and optionally substituted (C₁-C₆)alkenyl; and R⁶ is selected from the group comprising H, optionally substituted (C₁-C₆)alkyl, and optionally substituted (C₁-C₆)alkenyl, and optionally substituted heterocycle;
(c)

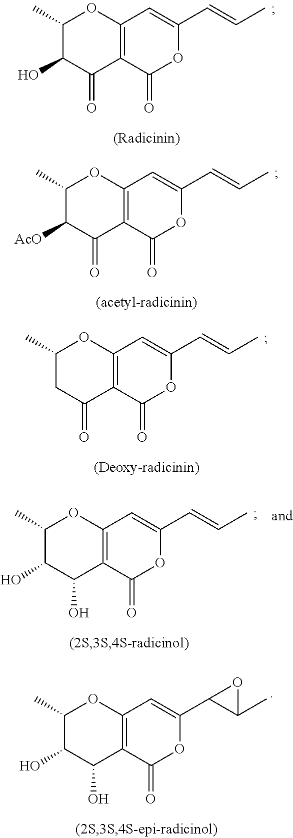

In another or further embodiment, the anti-Xf agent is administered by a syringe into the xylem.

The disclosure also provides a method of preparing an environment for growth of a plant susceptible to Xylella infection comprising (i) inoculating Pseudomonas sp. and/or Achromobacter sp. onto or into the soil or water, and/or (ii) inoculating an extract comprising an anti-Xf agent that inhibits Xylella sp. infection into the soil or water; and planting the plant.

The disclosure also provides a method of treating a Xylella infection in a plant comprising inoculating (i) Pseudomonas sp. and/or Achromobacter sp. into the xylem of the plant, and/or (ii) inoculating an extract comprising an anti-Xf agent that inhibits Xylella sp. infection into the xylem of the plant.

### BRIEF DESCRIPTION OF THE DRAWINGS

FIG. 1A-B show inhibition of Xylella fastidiosa with different fungal cultures. (A) Both Geomyces sp. and Achromobacter sp. suppressed Pierce Disease (PD) development in grapevine cuttings mechanically inoculated with Xf. Following inoculation, plants were rated weekly on a 0-5 disease scale with 0=healthy and 5=dead. (B) Geomyces sp., Achromobacter sp., and Aureobasidium sp. caused a reduction in Xf titer as compared to plants inoculated with Xf alone.

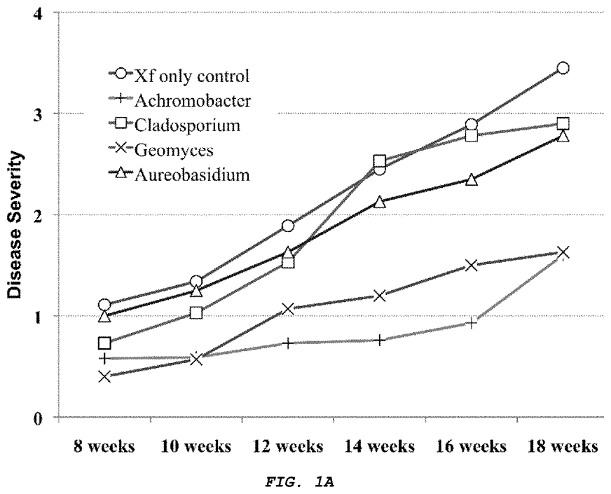

FIG. 2A-D shows relative abundance (percent) of the bacterial taxa inhabiting the grape vascular system endosphere: phylum (A), class (B) order (C), and genus (D). The operational taxonomic units (OTUs) that did not occur in at least 1% of the dataset were filtered out and the remaining OTUs are presented in relative abundance (N=72).

FIG. 3 shows abundance of genus level taxa identified in certified Xylella fastidiosa-free, PD-escape and PD-symptomatic vines (n=72).

FIG. 4A-B shows (A) Xylella fastidiosa relative abundance (mean percent) in PD-escaped and PD-symptomatic grapevines; ** indicates a statisitcal significant difference (P<0.01; n=67). (B) X. fastidiosa incidence (percentage of vines where X. fastidiosa reads could be detected) in PD-symptomatic versus PD-escape; ** indicates a statistical significant difference (P<0.01; n=67).

FIG. 5A-D shows relative abundance (percent) of the fungal taxa inhabiting the grape vascular system endosphere: phylum (A), class (B), order (C), and genus (D). The operational taxonomic unites (OTUs) that did not occur in at least 1% of the dataset were filtered out and the remaining OTUs are presented in relative abundance (N=34).

FIG. 6 shows Pseudomonas fluorescens relative abundance in PD-escaped (close column) and PD-symptomatic (open column) grapevines. The error bars represent 1 SE. *, FDR P<0.05; n=68.

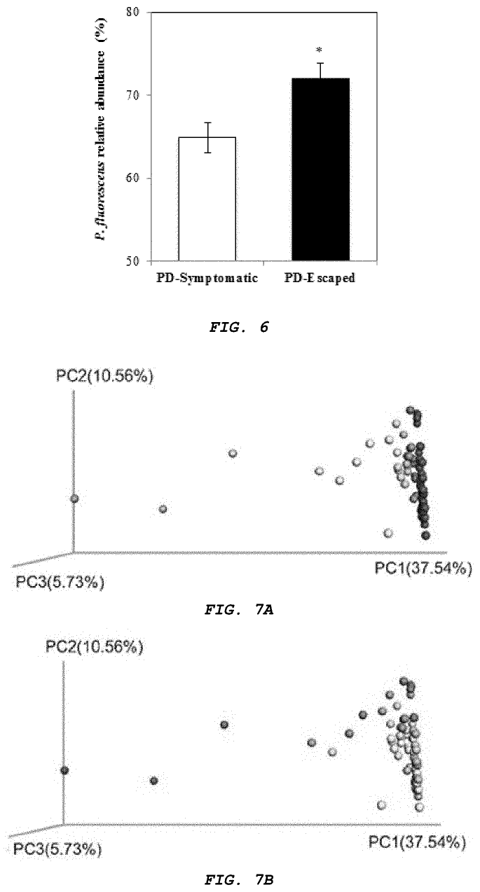

FIG. 7A-C shows principal coordinates analysis (PCoA) plots based on Hellinger distance matrix plotted with the first three dimensions. Comparison of bacterial communities by (A) abundance of Xylella fastidiosa and (B) abundance of Pseudomonas. Points represent individual samples in the data set. Dark gray dots represent higher abundance and light gray represent lower abundance. Adonis test revealed a significant P value and coefficient correlation for abundance of both Pseudomonas (P<0.001; R2−0.28) and Xylella P<0.001, R2=34). (C) shows bacterial community variation represented by canonical analysis of principal coordinates (Hellinger distance matrix) plotted on the first two dimensions. Top contributors of community variation are represented by arrows, Pf=Pseudomonas fluorescens; Xf=Xylella fastidiosa. Arrows are in divergent directions (P<0.001). Dots are colored by condition but the data were analyzed by X. fastidiosa abundance.

FIG. 8 shows Molecular Phylogenetic analysis by Maximum Likelihood method. The tree with the highest log likelihood (−4089.1849) is shown. The percentage of trees in which the associated taxa clustered as shown in this tree is designated at the nodes. The tree is drawn to scale, with branch lengths measured in the number of substitutions per site.

FIG. 9A-D shows Venn Diagrams depicting the number of shared and different operational taxonomic units based on four variable: (A) grapevine cultivar (n=60); (B) sampling year (n=72); (C) vineyard location (n=72); and (D) grapevine condition (n=72).

### DETAILED DESCRIPTION

As used herein and in the appended claims, the singular forms “a,” “and,” and “the” include plural referents unless the context clearly dictates otherwise. Thus, for example, reference to “a plant” includes a plurality of such plants and reference to “the pathogen” includes reference to one or more pathogens known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Crop infestations can cause severe damage to crop production and can cause severe economic harm to farmers and consumers. One bacterium responsible for plant infections is Xylella, such as Xylella fastidiosa (Xf). Xylella fastidiosa is a gram-negative, xylem-limited bacterium capable of affecting economically important crops. The bacterium has a large host range, including at least 28 families of both monocotyleyledonous and dictotyledonous plants. Plant hosts for X. fastidiosa include miscellaneous ornamentals, grape, oleander, oak, almond, peach, pear, citrus, coffee, maple, mulberry, elm, sycamore, and alfalfa, where the bacterium inhabits the plants' xylem. Other strains of Xylella cause important diseases of peach, citrus, coffee, and numerous forest tree species. Vectors, such as insects like xylem sap-feeding sharpshooters, acquire the bacterium by feeding on infected plants and subsequently infect other plants. Xylella can also be graft transmitted.

Pierce's Disease (PD) is caused by the Gram-negative, xylem-limited bacterium Xylella fastidiosa and is one of the major threats to grapevine production in the Americas. In addition, X. fastidiosa has recently been reported in olives in Italy and ornamentals in France iterating the importance of X. fastidiosa as a global re-emerging pathogen. Pierce's Disease (PD) is caused by the bacterium Xylella fastidiosa. X. fastidiosa is exclusively vectored by xylem-feeding insects belonging to the families Cicadellidae and Cercopidae, primarily sharpshooters. Insecticide sprays are the primary means of management of the disease aside from severe pruning or rogueing of infected vines. Currently, there are no sustainable control measures for PD and, hence, this disease remains a serious threat to California and worldwide grape production, and results in millions of dollars of annual economic losses related to damage to existing vineyard plantings, vector and disease management, and replanting. The bacterium resides in the xylem. Insects that feed on xylem sap transmit the bacteria from diseased plants to healthy plants and thus are the vector for spread of the disease and bacteria. Plants (e.g., grapevines) shows symptoms of bacterial and infection when the bacteria block the water conducting system and reduce the flow of water to the leaves. The first evidence of PD infection usually is a drying or “scorching” of leaves. About mid-growing season, when foliar scorching begins, some or all of the fruit clusters may wilt and dry up. The bark on affected canes often matures unevenly, leaving islands of mature (brown) bark surrounded by immature (green) bark or the reverse. Environmental stress (e.g., drought) hastens an infected plant's demise.

Pierce's Disease occurs in North America through Central America and has been reported in some parts of northwestern South America and is common in some California vineyards every year, with the most dramatic losses occurring in the Temecula Valley, Coachella Valley, Napa Valley and in parts of the San Joaquin Valley. Economic damages from the disease have been estimated to cost as much as $20,000 per acre. During severe epidemics, losses to PD may require major replanting. Currently there are more than 500 million commercial grapevines in the United States, with 40% of the acreage at risk for significant economic loss. Outbreak of Pierce's Disease in California has also had a major impact on the state's nursery business due to quarantines imposed in efforts to prevent the spread of the disease. In PD-impacted vineyards, there are observations of vines that remain asymptomatic despite being under high disease pressure.

The manifestation of disease is related to at least two parameters: (1) the successful multi-directional dissemination of Xf within the plant and (2) the plant phenotype that allows a particular strain of Xf to reach high populations within it. These parameters are associated and largely determine whether a particular strain of Xf will be a harmless entophyte or a pathogen in any given plant. Plant phenotype and the level of affinity each strain of Xf has for a particular plant species/strain are the primary factors relating to the disease causing threshold.

Dissemination of Xf within plants is by three modes: Horizontal movement by secreting pectin degrading enzymes to breaks down the pith membrane of xylem vessel walls allowing Xf to disseminate into adjacent xylem vessels. Xf is also carried along in the xylem flow to disseminate upward, e.g., away from the roots. The final mode of dissemination is for Xf to crawl against the flow of xylem. Xf uses minute tentacles (pili) to preferably and arduously migrate against the flow of xylem. This mode of dissemination is used by the bacterium to colonize the enduring regions of the plant, e.g., lower branches, trunk, and roots and thereby help insure the survival of Xf.

Once Xf colonizes the enduring regions of the plant such as the trunk, the infection tends to become chronic. Conversely Xf does not, of necessity, create a chronic infection when it infects a plant. If the bacterium does not achieve colonization of enduring regions of the plant and is limited to the green parts such as the canes that are pruned after harvest then the infection is removed.

Since the mid-1970s, other strains of Xylella fastidiosa have been discovered, and almost all of these cause leaf scorching of woody perennials, such as American elm, maple, mulberry, or plum. In some plants, such as peach and alfalfa, the bacterium slows and stunts plant growth. Xylella sp., such as Xylella fastidiosa, are responsible for variegated chlorosis in citrus, almond leaf scorch disease, phony peach disease, alfalfa dwarf, and others. Xylella fastidiosa attacks citrus fruits by blocking the xylem, resulting in juiceless fruits of no commercial value.

Methods of treating or preventing infection by Xylella which have been tried include control of the insect vectors (such as through pesticide and use or physical barriers), destruction of infected plants, and pruning and freezing. Other methods contemplated include the use of other bacterial species and bacteriophages, for the control of Xylella fastidiosa in host plants, the use of broad-spectrum antibiotics or boosting levels of essential plant bacterial micronutrients such as zinc, iron, copper, and molybdenum that could be toxic to Xylella sp. For example, U.S. Patent Application Publication No. 20050053584, describes the use of a benign strain of Xf (EB92-1) to inhibit a virulent strain of Xf. This form of bio-control can be referred to as “competitive displacement.” The deployment of competitive displacement is problematic, however, because the characteristics that make a particular strain virulent tend to give the virulent strain competitive advantage over the benign strains. Virulence by any one Xf strain is not universal to all plants that it infects; rather it is determined by the success of a particular Xf strain in its specific species/strain of host plant. Thus, when a strain of Xf comes to reside in its preferred plant host any benign strains of Xf that cohabitates the plant are at competitive disadvantage. The pre-establishment of the benign strain of Xf in the target plant may offset the benign strains disadvantage. This “head start advantage” tends to weaken over time and eventually the benign strain will yield to the aggressive strain. Thus, it may be required to reapply the benign strain after three to five years.

Yet another way to prevent the infection is by genetically modifying the chemistry and structure of the xylem making it uninhabitable for the bacteria (see, e.g., U.S. Pat. No. 6,232,528).

The disclosure provides evidence that the grapevine cane endosphere hosts a bacterial community structure with species of similar relative abundances to communities described for other annually produced aerial plant parts (i.e., grape flowers berries and leaves) with a high abundance of Proteobacteria and a low incidence of Firmicutes, Acidobacteria, and Bacteriodetes (Zarraonaindia et al. 2015). Faist et al. (2016) also supported these results although they found a higher abundance of Firmicutes, Actinobacteria, and Bacteriodetes in canes likely due to the difference with sampling location and design, grapevine cultivar and rootstock and environmental conditions. In addition, the disclosure demonstrates that Verrucomicrobia was represented in the cane endosphere, albeit low abundance. Zarraonaindia et al. (2015) showed that Verrucomicrobia were absent in leaves, berries, or flowers but were abundant in the soil, grape rhizosphere, and root endosphere. This suggests that the Verrucomicrobia found in the internal cane endosphere is likely derived from the soil or rhizosphere.

Fungi are an important segment of the plant microbiome and a key determinant to plant health. For example, fungi produce a diversity of structurally distinctive compounds with antimicrobial properties. Culture-independent grape microbiome studies have primarily been centered on bacterial community composition and this disclosure provides a description of culture-independent analyses of the fungal communities associated with grapevine cane endosphere. The disclosure shows that overall the fungal community composition was more diverse than the bacterial community composition in terms of total number of OTUs. Several taxa (Cryptococcus, Alternaria, Sporomiella, and Aureobasidium) inhabiting the grape cane endosphere were also previously reported in the grape phyllosphere (Perazzolli et al. 2014; Pinto et al. 2014). In addition, the disclosure shows that several fungal taxonomic groups remained unclassified suggesting that the conditions of the plant vascular system (i.e., low oxygen, negative pressure, and nutrient availability) have selected for unique taxa that have adapted to this particular niche that are not well represented in publicly available databases.

Computational analyses revealed that several fungal taxa correlated positively with X. fastidiosa either individually or as consortia and among these Aspergillus, Chaetomium and Alternaria emerged as the most common taxa. In addition, several fungal taxa (Diplodia, Dothiorella, Eutypa, Neofusicoccum) known to cause grapevine vascular diseases (Rolshausen et al., 2010; Urbez-Torres, 2011) were also identified as positive correlates with PD. Botryopshaeria fungi (i.e., Diplodia, Dothiorella and Neofusicoccum) are thought to be endophytes of trees that become pathogenic under stress conditions (Desprez-Loustau et al., 2006; Alvarez-Loayza et al., 2011; Paolinelli-Alfonso et al., 2016). However, it is unclear if the plants were predisposed to infection by these fungi because they were stressed by X. fastidiosa infection or if these fungi act as facilitators of PD.

At the genus and species level, the cane endosphere bacterial community was composed primarily of Pseudomonas. More particularly the disclosure shows that P. fluorescens was the main species inhabiting the grapevine cane endosphere and that it was in significantly higher abundance in PD-escape vines versus PD-symptomatic vines. In fact, the clustering of bacterial communities appeared to be driven by the incidence of these P. fluorescens and X. fastidiosa in the endosphere. This shows that P. fluorescens has the ability to mitigate the establishment of X. fastidiosa and confer the host escape phenotype. P. fluorescens is commonly found in association with the rhizosphere, phyllosphere and endosphere of other plant species. Moreover, it is a known biological control agent in many pathosystems, but can also protect against abiotic stresses and stimulate plant growth (Bardas et al. 2009; Lugtenberg and Kamilova 2009; Maksimov et al. 2011; Olanya et al. 2016; Samavat et al. 2014; Shen et al. 2013; Srinivasan et al. 2009). This bacterium produces a diverse array of secondary metabolites that have antimicrobial properties, such as phenazines, lipopeptides, hydrogen cyanide and 2,4-diacetylphloroglucinol. Antibiosis is also described as a mechanism underlying how this bacterium imparts broad spectrum disease control (Arseneault et al. 2016; Loper et al. 2012; Michelsen et al. 2015; Rezzonico et al. 2007). In addition, P. fluorescens is a well-described plant growth promoting rhizobacteria. It has been shown to actively colonize the plant rhizosphere and the root endosphere and activate ISR (Kloepper et al. 1992) that can confer increased immunity against a broad range of plant pests and diseases in some plant species (Bolwerk et al. 2003; Cabanas et al. 2014; Maksimov et al. 2011; Pieterse et al. 1996; Pieterse et al. 2014). In grapevine, P. fluorescens can become established in the grape rhizosphere and suppress the growth of the pathogenic bacterium Agrobacterium spp., the causal agent of crown gall (Khmel et al. 1998). Moreover, it can actively colonize the root endosphere and trigger a systemic induced response that provide enhanced resistance against the leaf and berry pathogen Botrytis cinerea, the causal agent of gray mold (Aziz et al. 2016; Gruau et al. 2015).

Achromobacter xylosoxidans was also an abundant member of the microbial community occupying the grape cane endosphere that correlated negatively with X. fastidiosa suggesting that this bacterial taxon could also play a role in the disease escape phenotype. A. xylosoxidans is a known plant growth promoting rhizobacteria (Forchetti et al. 2010; Mayak et al. 2004) and a biological control agent of many plant pathogens (de Boer et al. 2015; Triki et al. 2012). A. xylosoxidans is commonly found in aqueous environments and soils. This organism can metabolize nitrate and glyphosate (Ermakova et al. 2010; Felgate et al. 2012), which are commonly used in grape production systems as part of fertilization regimes and weed management, respectively.

Plants can acquire microorganisms from soil and those selected microbes can move to the rhizosphere and root endosphere to fulfill specific biological functions (Berendsen et al. 2012). It was also proposed that organisms that inhabit the vascular system originate in part from the roots through the lumen of xylem vessels via the plant evapotranspiration stream (Compant et al. 2010). Other entry points to the plant vascular system may also include insect feeding (Lopez-Fernandez et al. 2017) wounding (Munkvold and Marois 1993) and perhaps plant natural openings (i.e., stomata).

The disclosure demonstrates that P. fluorescens and was found in significantly higher abundance in PD-escape vines vs. PD-symptomatic vines.

PiCrust analysis of the grapevine cane endosphere microbiome predicted that bacterial Type VI secretion systems are prevalent in this ecological niche. Type VI secretion systems are found in many Pseudomonads and can deliver antimicrobial protein effectors into target cells (Barret et al., 2011; Records, 2011). Thus, Type VI secretion systems are thought to play a key role in inter-microbial interactions. Specifically, some T6SS effectors, such as Tse2, secreted by Pseudomonas aeruginosa can act as toxins that are antimicrobial to other species of bacteria. Pseudomonas spp. and Burkholderia spp. use their T6SSs to compete with other bacteria. All sequenced genomes of Pseudomonas spp. contain one or more T6SS (Barret et al., 2011) and the primary phylotype that contributed to the abundance of genes associated with the T6SS system in the grapevine endosphere belongs to the Pseudomonadales. The antimicrobial effects of the Type VI secretion system require direct cell-cell contact and are mediated through a secretion apparatus that is thought to have been co-opted from a bacteriophage. Thus, it is speculate that any Type VI-mediated inhibitory interactions against X. fastidiosa in planta would be important early on in the infection process when X. fastidiosa is initially introduced into the xylem by its insect vector. These cells would be just beginning to adhere to the xylem wall and not housed in a 3-dimensional mature X. fastidiosa biofilm where it would be difficult for exogenous bacterial species to accomplish direct cell-cell contact with the bulk of the X. fastidiosa biofilm population.

Iron acquisition is another important modulator of interspecies competition and the PiCrust analyses revealed that genes encoding siderophore biosynthetic genes are prevalent in the grapevine cane endosphere indicating that iron competition is important for endosphere colonization. X. fastidiosa possesses several putative ferric enterobactin-like receptors (Simpson et al., 2000; Van Sluys et al., 2003) but, interestingly, genes encoding its own siderophores are apparently absent. This suggests that X. fastidiosa co-opts the siderophores of other organisms to acquire its iron. Because iron is likely a limited resource in planta, X. fastidiosa may have evolved a clever social cheating mechanism to compete for iron in the xylem.

The disclosure thus provides methods and compositions useful for treating or inhibiting infection by X. fastidiosa in plants. The method and compositions can comprise one or more biological agents delivered to the plant or soil or water in which the plant grows. The biological agent can comprise one or more of Pseudomonas florescens, Achromobacter xylosoxidans, Aspergillus sp., Cochliobolus sp., Cryptococcus sp., Discostroma sp., Geomyces sp., Phaeosphaeria sp., Ulocladium sp.

This disclosure can use Pseudomonas sp. and/or Achromobacter sp. in combination with other microorganisms that inhibit and treat Xf infection as well as compounds that inhibit, cure and/or treat Xf infection. Such bacteria possess anti-Xf properties, likely due to the production of certain metabolites.

Seven fungi have been identified that were strongly inhibitory to Xf in vitro, all of which have been identified to genus level based on ribosomal DNA sequences and include Aspergillus sp., Cochliobolus sp., Cryptococcus sp., Discostroma sp., Geomyces sp., Phaeosphaeria sp., and Ulocladium sp. In addition, and as mentioned above, two major bacterial phylotypes inhabiting the grape cane endosphere (i.e., Pseudomonas and Achromobacter) correlated negatively with X. fastidiosa abundance were identified.

Using the methods described above and elsewhere herein, anti-Xf endophytic microorganisms and fungi were identified that are useful for treating plants having or susceptible to an Xf infection as well as preventing spread or infection by Xf. As used herein the term “anti-Xf endophytic microorganisms” refers to two major bacterial phylotypes inhabiting the grape cane endosphere (i.e., Pseudomonas and Achromobacter), and/or the fungal phylotypes of Aspergillus sp., Cochliobolus sp., Cryptococcus sp., Discostroma sp., Ulocladium sp., Phaeosphaeria sp., Geomyces sp. and Achromobacter sp.

The anti-Xf endophytic microorganisms of the disclosure and anti-Xf agents isolated therefrom are useful in the treatment of target plants to confer Xf resistance and/or treatment of infection. Suitable plants include, but are not limited to, ornamentals, grape, olive, oleander, oak, almond, peach, pear, citrus, coffee, maple, mulberry, elm, sycamore, alfalfa, peach, and numerous forest tree species susceptible to Xylella sp. infection such as Xf infection. The plant may be at any stage of growth, including seeds, seedlings, or full plants. In addition, as discussed herein, any part of the plant or the soil or water may be inoculated; suitable plant parts include seeds, roots, leaves, flowers, stems, trunks, etc. Typically, the delivery will be to the endosphere or trunk of the plant.

In one embodiment the disclosure relates to target plants obtained by artificially introducing an anti-Xf endophytic microorganisms into plants and plant parts not infected with an anti-Xf endophytic microorganisms. In the context of this disclosure, the anti-Xf endophytic microorganisms which is artificially introduced into the target plant, is a microorganism that confers Xf infection resistance to the target plant. For example, the method of the disclosure includes delivery of P. florescens and/or A. xylosoxidans to a plant or environment lacking the presence of such microorganism. In one embodiment, the method includes delivery of P. florescens and/or A. xylosoxidans to a plant or environment having a low level of such microorganisms or into an environment or plant having Xf.

In a one embodiment, a pure culture of an anti-Xf endophytic microorganism is used to inoculate plants or plant parts. A “pure culture” in this context means a culture devoid of other cultured endophytic fungi. The culture will typically comprise a suitable carrier that is non-toxic to plants. Where an anti-Xf endophytic microorganism is a fungal organism or certain bacterial species, the culture may be of spores, hyphae, mycelia, or other forms of the fungi, with spores typical. In general, spores are used at 1-5×10³⁻⁸ spores per plant with 1-3×10⁴⁻⁶ being common and 1-3×10⁵ being typical. As outlined herein, the anti-Xf endophytic microorganisms of the disclosure may be cultured in a variety of ways, including the use of plates and liquid cultures.

In another embodiment, the plant may be inoculated by vacuum infiltration. In this embodiment, a stem or cutting is placed in an aqueous media comprising the anti-Xf endophytic microorganisms and a vacuum is applied to draw the aqueous media comprising the anti-Xf endophytic microorganisms into the cutting or stem. Once inoculated the cutting or stem is grown using recognized techniques. Typically this process is performed in a nursery.

The spores or other innoculum may be placed on seed coats, particularly on seeds of anti-Xf endophytic microorganisms-free seeds (either naturally occurring or treated to remove any endophytes). It should be noted that the plants, including seeds, may be inoculated with combinations of anti-Xf endophytic microorganism cultures.

In another embodiment, an extract of an anti-Xf endophytic microorganism comprising an agent that inhibits Xf growth or infection is used to treat or inhibit a Xylella sp. infection (e.g., a Xf infection that causes PD). For example, an extract from an anti-Xf endophytic microorganism can comprise an agent that inhibits Xf growth and transmission. In this embodiment, a culture of an anti-Xf endophytic microorganism can be disrupted and crude or purified extract may be applied to the plant. In one embodiment, the extract or purified composition is administered to the plant at the site of a Xylella sp. growth (e.g., the trunk or stem).

For example, in one embodiment, an extract of an anti-Xf endophytic microorganism of the disclosure was prepare and purified to identify an agent that inhibits Xf growth. The agent was identified as having structural Formula I:

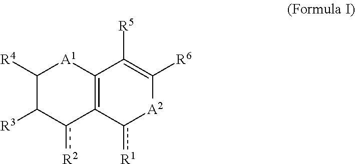

wherein,

A¹ and A² are each individually O, S, or NH;

R¹ and R² are each individually selected from the group comprising halo, CN, O, OH, NH, NH₂, S, SH, and CH₂;

R³-R⁵ are each individually selected from the group comprising H, halo, hydroxyl, cyano, thiol, amino, optionally substituted (C₁-C₆)alkyl, and optionally substituted (C₁-C₆)alkenyl; and

R⁶ is selected from the group comprising H, optionally substituted (C₁-C₆)alkenyl, and optionally substituted (C₁-C₆)alkenyl, and optionally substituted heterocycle.

In a further embodiment, the disclosure provides for a compound having structural Formula Ia:

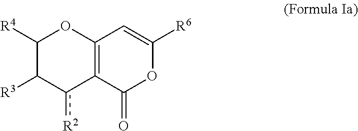

wherein,

R² is selected from the group comprising halo, CN, O, OH, NH, NH₂, S, SH, and CH₂;

R³-R⁴ are each individually selected from the group comprising H, halo, hydroxyl, cyano, thiol, amino, optionally substituted (C₁-C₆)alkyl, and optionally substituted (C₁-C₆)alkenyl; and

R⁶ is selected from the group comprising H, optionally substituted (C₁-C₆)alkyl, and optionally substituted (C₁-C₆)alkenyl, and optionally substituted heterocycle.

In another embodiment, the disclosure provides for a compound having structural Formula Ib:

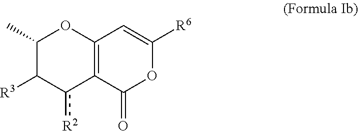

wherein,

R² is selected from the group comprising halo, CN, O, OH, NH, NH₂, S, SH, and CH₂;

R³ is individually selected from the group comprising H, halo, hydroxyl, cyano, thiol, amino, optionally substituted (C₁-C₆)alkyl, and optionally substituted (C₁-C₆)alkenyl; and

R⁶ is selected from the group comprising H, optionally substituted (C₁-C₆)alkyl, and optionally substituted (C₁-C₆)alkenyl, and optionally substituted heterocycle.

In a specific embodiment, the agent is selected from the group consisting of:

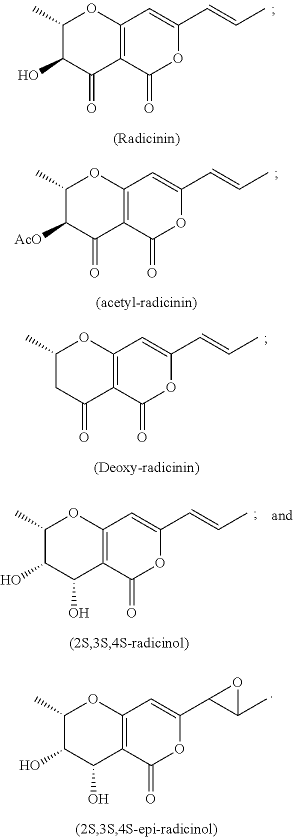

As used herein an anti-Xf agent of the disclosure refers to an agent having structural Formula I:

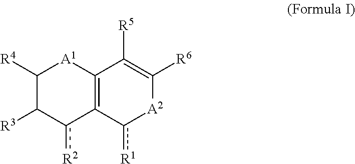

wherein,

A¹ and A² are each individually O, S, or NH;

R¹ and R² are each individually selected from the group comprising halo, CN, O, OH, NH, NH₂, S, SH, and CH₂;

R³-R⁵ are each individually selected from the group comprising H, halo, hydroxyl, cyano, thiol, amino, optionally substituted (C₁-C₆)alkyl, and optionally substituted (C₁-C₆)alkenyl; and

R⁶ is selected from the group comprising H, optionally substituted (C₁-C₆)alkyl, and optionally substituted (C₁-C₆)alkenyl, and optionally substituted heterocycle.

In a further embodiment, the disclosure provides for an anti-Xf agent having structural Formula Ia:

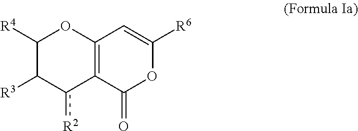

wherein,

R² is selected from the group comprising halo, CN, O, OH, NH, NH₂, S, SH, and CH₂;

R³-R⁴ are each individually selected from the group comprising H, halo, hydroxyl, cyano, thiol, amino, optionally substituted (C₁-C₆)alkyl, and optionally substituted (C₁-C₆)alkenyl; and

R⁶ is selected from the group comprising H, optionally substituted (C₁-C₆)alkyl, and optionally substituted (C₁-C₆)alkenyl, and optionally substituted heterocycle.

As used herein an anti-Xf agent of the disclosure refers to an agent having structural Formula Ib:

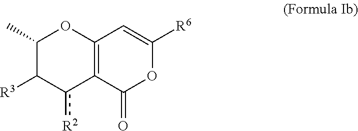

wherein,

R² is selected from the group comprising halo, CN, O, OH, NH, NH₂, S, SH, and CH₂;

R³ is individually selected from the group comprising H, halo, hydroxyl, cyano, thiol, amino, optionally substituted (C₁-C₆)alkyl, and optionally substituted (C₁-C₆)alkenyl; and

R⁶ is selected from the group comprising H, optionally substituted (C₁-C₆)alkyl, and optionally substituted (C₁-C₆)alkenyl, and optionally substituted heterocycle.

As used herein an anti-Xf agent of the disclosure refers to an agent selected from the group consisting of:

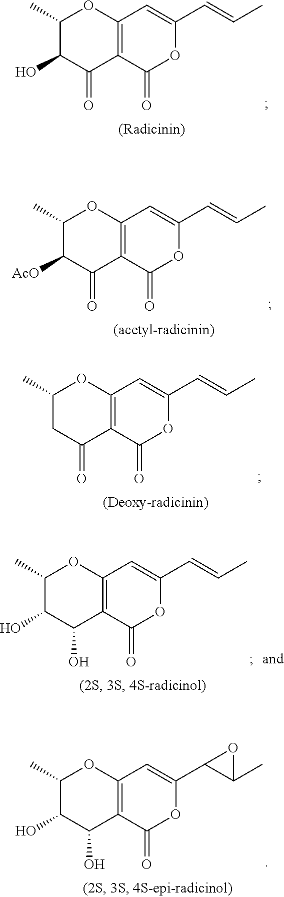

In one embodiment, a combination of anti-Xf agents or an anti-Xf agent and an anti-Xf endophytic microorganism may be used to treat an Xf infection or prevent an Xf infection.

Methods are provided for protecting a plant from a pathogen (e.g., an Xf pathogen) comprising applying an effective amount of a composition comprising an anti-Xf endophytic microorganism of the disclosure and/or an anti-Xf agent/compound of the disclosure. “Effective amount” is intended to mean an amount sufficient to control a virulent pathogen.

The compositions comprising the anti-Xf endophytic microorganism(s) and/or an anti-Xf agent of the disclosure may comprise a surface-active agent, an inert carrier, a preservative, a humectant, a feeding stimulant, an attractant, an encapsulating agent, a binder, an emulsifier, a dye, a UV protective, a buffer, a flow agent or fertilizers, micronutrient donors, or other preparations that influence plant growth. One or more agrochemicals including, but not limited to, herbicides, insecticides, nematicides, molluscicides, acaracides, plant growth regulators, harvest aids, and fertilizers, can be combined with carriers, surfactants or adjuvants customarily employed in the art of formulation or other components to facilitate product handling and application for particular target pathogens. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the disclosure are normally applied in the form of compositions and can be applied to the crop area, plant, or seed to be treated. For example, the compositions of the disclosure may be applied during growth, seeding or storage.

The anti-Xf endophytic microorganism(s) and/or an anti-Xf agent of the disclosure may be applied simultaneously or in succession with other compounds. Methods of applying a composition of the disclosure include, but are not limited to, foliar application, seed coating, injection and soil application. The number of applications and the rate of application depend on the intensity of infestation by the corresponding pathogen.

Suitable surface-active agents include, but are not limited to, anionic compounds such as a carboxylate of, for example, a metal; carboxylate of a long chain fatty acid; an N-acylsarcosinate; mono or di-esters of phosphoric acid with fatty alcohol ethoxylates or salts of such esters; fatty alcohol sulfates such as sodium dodecyl sulfate, sodium octadecyl sulfate or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitar fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include, but are not limited to, inorganic minerals such as kaolin, phyllosilicates, carbonates, sulfates, phosphates, or botanical materials such as cork, powdered corncobs, peanut hulls, rice hulls, and walnut shells.

The compositions of the disclosure can be in a suitable form for direct application or as a concentrate of primary composition that requires dilution with a suitable quantity of water or other diluent before application. The concentration of benign fungal organism will vary depending upon the nature of the particular formulation, specifically, whether it is a concentrate or to be used directly.

A compositions of the disclosure can be applied to the environment of a plant pathogen by, for example, spraying, atomizing, dusting, scattering, coating, injecting or pouring, introducing into or on the soil, introducing into irrigation water, by seed treatment or general application or dusting at the time when the pathogen has begun to appear or before the appearance of pathogens as a protective measure. It is generally important to obtain good control of pathogens in the early stages of plant growth, as this is the time when the plant can be most severely damaged. The compositions of the disclosure can conveniently contain an insecticide if this is thought necessary.

In one embodiment, an anti-Xf agent and/or anti-Xf-microorganism of the disclosure can be injected into the trunk of a plant (e.g., a grape vine). For example, an 18-gauge syringe needle can be attached to a syringe barrel and used to pierce the base of the vine with the needle. The injection apparatus can be secured to the vine and left in place. This will allow for a continuous drip of the solution into the xylem stream of the plant—the niche where Xf dwells. This xylem infiltration technique is used by arborists to deliver insecticides, fungicides or plant growth regulators to achieve systemic translocation of the product into the xylem stream. There are several commercial products built on this concept, including SilvaShield Injectable (Bayer AG), Merit Injectable (Tree Tech, Inc.) and ChemJet Tree Injector (ChemJet).

Compositions of the disclosure find use in protecting plants, seeds, and plant products in a variety of ways. For example, the compositions can be used in a method that involves placing an effective amount of the composition in the environment of the pathogen by a procedure selected from the group consisting of spraying, dusting, broadcasting, or seed coating. The methods of are effective against a variety of plant pathogens.

### EXAMPLES

### Example 1

Plant Sample Collection.

Samples were collected from 10 vineyards in California (Table 1). Five vineyards var. Syrah, Petite Sirah, Chardonnay, Tempranillo, and Mourvedre were located in Temecula Valley, in southern California. This hot and arid viticulture area has been severely impacted by PD, because of the introduction of the invasive glassy winged sharpshooter (GWSS), Homalodisca vitripennis (Blua et al., 1999). In addition, four vineyards var. Merlot, Riesling and Chardonnay (two vineyards) were located in Napa Valley in northern California. This viticultural area is wetter and cooler than the southern area. In northern California, X. fastidiosa is commonly vectored by the California native blue-green sharpshooter (BGSS), Graphocephala atropunctata. Historically, the incidence of PD in northern California vineyards has been primarily isolated to vineyards adjacent to riparian areas, which serve as reservoirs of the BGSS (Hopkins & Purcell, 2002). These areas have high incidences of PD and are referred to as PD hotspots. Because of the distinctly different feeding and breeding behavior of the GWSS in southern California, entire vineyards are impacted with severe PD rather than being isolated to hotspots (Perring et al., 2001). In northern California, disease escape and infected vines were sampled from within PD hotspots and in southern California disease escape and infected vines were sampled from within vineyards highly impacted with PD (adjacent to citrus groves that serve as reservoirs for the GWSS). Grapevine tissue was sampled over 3 years at each field site in August/September, when grapevines express typical PD symptoms. A total of 68 samples were collected from 9 vineyards in apa or emecula (Table 1), with half from grapevines expressing PD symptoms (PD-symptomatic) and the other half from healthy grapevines (PD-escape). Canes were randomly selected from PD-symptomatic grapevines and from PD-escape grapevines. Following sampling, canes were stored in a cooler on ice and brought back to the laboratory. In addition, five one year-old grape cuttings var. Chardonnay (two samples) and Merlot (three samples) originating from the Foundation Plant Service (FPS) block at UC Davis were included in the pool of samples to build the core endosphere microbiome of grapevine canes, because this plant material is routinely tested for X. fastidiosa and certified disease-free. In the laboratory, one internode was harvested with sterile pruning shears from each cane. Internode samples were dipped in 100% ethanol and surface sterilized by flaming. Subsequently, the bark was removed with sterile blade and these de-barked samples were stored at −80° C.

TABLE 1
Scheme of the 73 grapevine cane samples collected.

| | Number of | | Year | Number of |
|---|---|---|---|---|
| Location | vineyards | Cultivar | collected | samples |
| Temecula | 5 | Chardonnay | 2013 | 10 |
| | | Mouvedre | 2013 | 10 |
| | | Petite Sirah | 2013 | 10 |
| | | Syrah | 2010 | 12 |
| | | Temranillo | 2013 | 10 |
| Napa | 4 | Chardonnay | 2011 | 8 |
| | | Merlot | 2011 | 4 |
| | | Riesling | 2011 | 4 |
| FPS Davis | 1 | Chardonnay | 2014 | 2 |
| | | Merlot | 2014 | 3 |

DNA Extraction from De-Barked Cane Tissue.

Frozen tissues were lyophilized for 36 h with a FreeZone 2.5 l Benchtop Freeze Dry System (Labconco, Kansas City, Mo.). Following this, the samples were finely chopped with sterilized knifes and ground at room temperature using a Retsch MM300 grinder (90 s, 25 oscillations per second) in a 35 ml stainless-steel grinding jar (Retsch, Haan, Germany) with 20 mm stainless steel balls as previously described (Pouzoulet et al., 2014). Total DNA was extracted from 50 mg of dried tissue using a Qiagen DNeasy plant mini kit (Qiagen, Valencia, Calif.) according to manufacturer's instructions with the exception of using CTAB (hexadecyltrimethylammonium bromide, Sigma-Aldrich, St. Louis, USA) extraction buffer (Doyle & Doyle, 1987) as the lysing buffer instead of the AP1 buffer provided with the kit. The DNA for all samples was aliquoted and stored at −20° C. before use.

Bacterial rRNA Gene Sequencing.

Illumina bacterial 16S rRNA gene libraries were constructed as previously described (Ruegger et al., 2014). 100 μl amplification reactions were performed in a MJ Research PTC-200 thermal cycler (Bio-Rad Inc., Hercules, Calif.) containing 50 mM Tris (pH 8.3), bovine serum albumin (BSA) at 500 μg/ml, 2.5 mM MgCl₂, 240 μM of each deoxynucleotide triphosphate (dNTP), 400 nM of each PCR primer, 4 μl of DNA template and 2.5 units of JumpStart Taq DNA polymerase (Sigma-Aldrich). Briefly, the PCR primers 515F (GTGCCAGCMGCCGCGGTAA—SEQ ID NO:1) and 806R (GGACTACHVGGGTWTCTAAT—SEQ ID NO:2) targeted the 16S rRNA gene containing portions of the hpervariable regions V4 and V5, with the reverse primers including a 12-bp barcode (Caporaso et al., 2010). Thermal cycling parameters were 94° C. for 20×, 50° C. for 20 s and 72° C. for 30 s, followed by 72° C. for 5 min. PCR products were purified using the MinElute 96 UF PCR Purification Kit (Qiagen).

DNA sequencing was performed using an Illumina MiSeq (Illumina, Inc, San Diego, Calif.). Clusters were created using template concentrations of 1.5 pM and PhiX at 65 K mm⁻² and 301 base read lengths were truncated after the 260^{th} base where sequence quality was diminished. Sequences were then processed using Quantitative Insights Into Microbial Ecology (QIIME) version 1.8.0 (Caporaso et al., 2010). De-multiplexing and quality filtering were performed using default parameters except that the minimum number of consecutive high quality base calls to include a read as a fraction of input read length was raised to 0.99. Chimeric sequences were detected using the denovo method of USEARCH 6.1 (Edgar, 2010) and then removed. Denovo OTUs were picked at 97% similarity and singlet OTUs were removed. OTUs having non-bacterial DNA were identified by performing a local BLAST search (Altschul et al., 1990) of their sequences against the nt database. OTUs were removed if any of their highest scoring BLAST hits contained taxonomic IDs within the grapevine family Vitaceae, fungi or PhiX, or if they contained no hits to any bacteria. Taxonomic assignments to bacterial OTUs were made using the May 5, 2013 version of the Greengenes reference database (DeSantis et al., 2006) using QIIME's default parameters. For statistical and graphical representation of the data, OUT tables were rarified to an even depth of 6,000 reads per samples. Samples with less than 6,000 reads were removed from the OUT table. The bacterial sequences can be found at NCBI under the accession number PRJNA361009.

Fungal rRNA Gene Sequencing:

Illumina fungal rRNA ITS libraries were constructed as follows. One hundred μl amplification reactions were performed in an MJ Research PTC-200 thermal cycler (Bio-Rad Inc., Hercules, Calif., USA) and contained 50 mM Tris (pH 8.3), 500 μg ml⁻¹ BSA, 2.5 mM MgCl₂, 250 μM of each deoxynucleotide triphosphate (dNTP), 400 nM of each primer, 4 μl of DNA template, and 2.5 units JumpStart Taq DNA polymerase (Sigma-Aldrich, St. Louis, Mo., USA). The PCR primers gITS7 (GTGARTCATCGARTCTTTG—SEQ ID NO:3) and ITS4 (TCCTCCGCTTATTGATATGC—SEQ ID NO:4) targeted the ITS2 region of the ribosomal rRNA gene operon, with the reverse primers including a 7-bp barcode (White et al., 1990; Ihrmark et al., 2012). Thermal cycling parameters were 94° C. for 5 min; 35 cycles of 94° C. for 20 s, 56° C. for 20 s, and 72° C. for 30 s, and followed by 72° C. for 10 min. PCR products were purified using a Qiagen QIAquick PCR purification kit (Qiagen, Valencia, Calif.) according to the manufacturer's instructions.

DNA sequencing was performed using an Illumina Miseq (Illumina, Inc., San Diego, Calif., USA). Clusters were created using template concentrations 1.0 pM and PhiX at 65 K/mm². Raw read lengths were 301 bases. Raw sequences were processed using QIIME version 1.8.0 (Caporaso et al., 2010). De-multiplexing and quality filtering were performed using default parameters except that the minimum allowed Phred quality score was raised to Q30. Chimeric sequences were detected using the denovo method of USEARCH 6.1 (Edgar, 2010) and then removed. Denovo OTUs were picked at 99% similarity and singlet OTUs were removed. OTUs having non-fungal DNA were identified by performing a local BLAST search (Altschul et al., 1990) of their sequences against the nt database. OTUs were removed if any of their highest scoring BLAST hits contained taxonomic IDs within the grapevine family Vitaceae or PhiX, or if they contained no hits to any fungi. Taxonomic assignments to fungal OTUs were made with the RDP Classifier (Wang et al., 2007) using the July, 2014 UNITE fungal database (Koljalg et al., 2005). BLASTN 2.4.0 was used to identify the top 50 genus level sequences that were unable to be identified through RDP database. Genus level names were adopted for each sequence if there was a 97% identity and had a cut-off E-value of E-120. The newly identified genera were added to the original OTU table obtained through QIIME. For fungal taxonomy barplots, OUT tables were rarefied to 1,000 reads per sample. Samples with less than 1,000 reads were removed from the dataset. The fungal sequences can be found at NCBI under the accession number PRJNA361126.

Microbiome Data Analyses.

Using the rarified OUT tables form QIIME, R 3.4.0 (see World Wide Web at (www).r-project.org/) was employed for the graphical and statistical analysis. The phyloseq package 1.20.2 (McMurdie and Holmes 2013) and ggplot2 2.2.1 (Wickham 2009) were utilized for further parsing the OUT tables and graphical representations. Taxonomy plots for bacterial and fungi were generated from 72 and 34 samples, respectively, by pruning OTUs for only those representing at least 1% of the bacterial or fungal OTU table. Venn diagrams were also created with the R package VennDiagram (Chen and Boutros 2011) version 1.6.17 ([https://]cran.rstudio.com/web/packages/VennDiagram/VennDiagram.pd f) and demonstrated the presence/absence of a number of bacterial OTUs occurring in various metadata categories including cultivar (60 samples), sampling year (72 samples), vineyard location (72 samples) and grapevine condition (72 samples). Wilcoxon test corrected by false discovery rate (FDR) values were used to statistically compare X. fastidiosa abundance and incidence in PD-symptomatic versus PD-escape grapevine. Pearson correlation analyses from 67 samples (excluding FPS plant samples) between the numbers of X. fastidiosa sequencing reads and the relative abundance of the other bacterial taxa were performed using the R package Hmisc 4.0-3 ([https://]biostat.mc.vanderbilt.edu/wiki/Main/Hmisc). FDR was used to adjust P values. Hellinger distance matrix was calculated using 72 samples with QIIME (Capraso et al. 2010) and used to create principal coordinates analysis (PCoA) plots. The Hellinger distance matrix was also used on all 72 samples to create the Canonical analysis of principal coordinates (CAP) plot using the Vegan package ([https://]cran.r-project.org/web/packages/vegan/index.html). Statistics for the PCoA was carried out with an Adonis test. Adonis was run on Xylella and Pseudomonas abundances. Similarly, ANOVA like permutations test for constrained analysis was run to determine statistical significance between Xylella and Pseudomonas abundance among samples.

Chromosome Primer Walking.

To obtain the sequences of a greater portion of the 16S rRNA gene along with the internal transcribed spacer (ITS) region for selected OTUs, chromosome walking was conducted as follows. Samples that showed relative high abundance of the most abundant Pseudomonas OTU#646559 and Achromobacter OTU#558264 based on Illumina sequencing were selected. PCR was performed on the total DNA using a nested approach. The first PCR used primers targeting the small-subunit rRNA gene and the large-subunit rRNA gene: 530F (GTGCCAGCMGCCGCGG—SEQ ID NO:5) and 23SR (GGGTTBCCCCATTCRG—SEQ ID NO:6). PCR products were diluted 1:99 and then subjected to sequence-selective PCRs where the reverse primer was 23SR (GGGTTBCCCCATTCRG—SEQ ID NO:6) and the following sequence-selective forward primers were: Achromobacter (TTTAACTACCGAGCTAGAG—SEQ ID NO:7), Opitutus (AACTGCGTCTGAAACTACAT—SEQ ID NO:8), Pseudomonas (AACTGACTGACTAGAGTAT—SEQ ID NO:9) and Sulfuritalea (AACTGCGTTTGTGACTGT—SEQ ID NO:10). Amplification products were gel isolated and cloned as described previously (Bent et al., 2009), and the nucleotide sequences were obtained using ABI Cycle Sequencing (Applied Biosystems, Foster City, Calif.). Sequence identities were determined by an analyses using BLAST (NCBI) (Altschul et al., 1997). For the Pseudomonas analyses, 16 different samples were run and obtained bands of the predicted size from 14 samples. Six of these bands were cloned and sequenced, resulting in eight sequences from three different samples that had 100% identity to the sequence of Illumina Pseudomonas OTU #646549. These sequences were examined further by a phylogenetic tree analysis described elsewhere herein. For the Achromobacter analyses, 16 different samples were run and obtained bands of the predicted size from 13 samples. Six of these bands were cloned and sequenced, resulting in four sequences from two different samples that had 100% identity to the sequences from Illumina Achromobacter OTU #558264. The best match to all four of these sequences was Achromobacter xylosoxidans as determined using BLAST analysis.

Phylogenetic Analysis.

To better identify the species name of the most abundant Pseudomonas OTU#646549, a multi-loci phylogenetic tree was created. BLASTN was used to find homologous sequences in the Pseudomonas genome sequence database published by Loper et al. (2012). The sequences averaged 1524 base pairs in length and included 16S, ITS and 23S loci. A total of 32 Pseudomonas sequences were aligned using ClustalW (Geneious v. 6.1.6; Biomatters Ltd.). The evolutionary history was inferred by using the Maximum Likelihood method based on the Tamura-Nei model (Tamura & Nei, 1993). Initial trees for the heuristic search were obtained automatically by applying Neighbor-Join and BioNJ algorithms to a matrix of pairwise distances estimated using the Maximum Composite Likelihood approach, and then selecting the topology with superior log likelihood value. All positions containing gaps and missing data were eliminated. There were a total of 1408 positions in the final dataset. Bootstrap values were inferred using 1,000 replicates. Evolutionary analyses were conducted in MEGA7 (Kumar et al., 2016).

The Core Endophytic Microbiome of Grapevine.

The grapevine endophytic microbiome was characterized from lignified shoot samples collected from ten vineyards across California, including five commercial vineyards in the Temecula valley, four commercial vineyards in the Napa Valley and one vineyard from the Foundation Plant Services at UC Davis in Davis, Calif. A total of 60 and 1.1% of the total number of reads were of grapevine origin for bacteria and fungi, respectively. After removing plants reads, 2,209,462 reads were obtained and 462,294 total high quality reads for the bacterial and fungal data sets. After processing the rarefied data, 918 and 4,437 bacterial and fungal OTUs were identified, respectively.

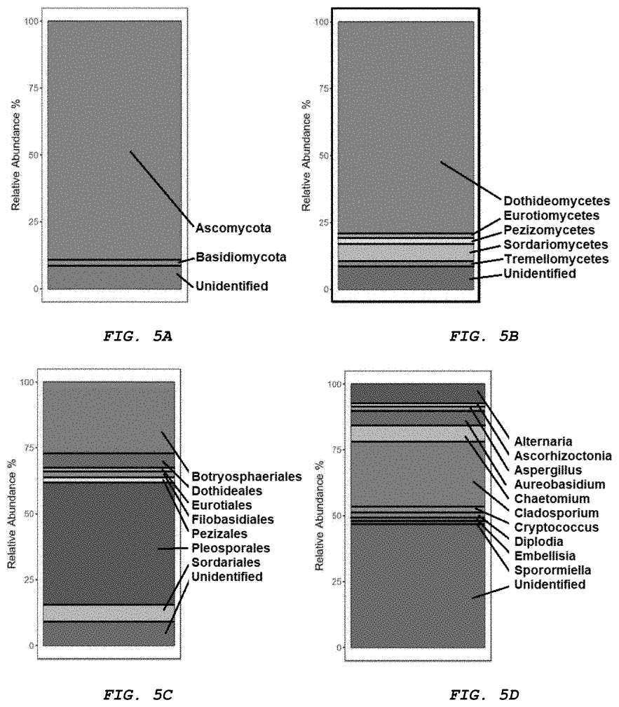

Community analysis was performed with only OTUs representing greater than 1% of the data and transformed to relative abundance for taxonomy plots. For fungal taxonomy, barplots (FIG. 5A-D) were constructed with 34 samples because 39 samples with less than 1,000 reads were removed from the dataset. The results showed that fungal communities were predominately comprised of phyla belonging to the Ascomycota (89.2%), which was mostly composed of four classes including Dothideomycetes (79.3%), Sordariomycetes (6.4%), Pezizomycetes (2.2%), and Eurotiomycetes (1.3%). Within the Dothidiomycetes, the Pleosporales (46.5%) was the most abundant order and was comprised of four genera including one unassigned genera Alternaria, Sporomiella, and Embellisia. The Dothidiomycetes also included three additional orders Capnodiales (24.5%), Dothideales (6%), and Botryosphaeriales (2.1%) and were represented predominantly by Cladosporium, Aureobasidium, and Diplodia, respectively. The Sordariomycetes was represented predominantly by the order Sordariales (6.4%) with one genus, Chaetomium. The Eurotiomycetes was represented predominantly by the order Eurotiales (1.3%) comprised of mainly Aspergillus. Pezizomycetes was represented by the single order Pezizales and one unassigned genus and the genus Ascorhizoctonia. Organisms belonging to the phylum Basidiomycota represented only 1.9% and were mostly composed of organisms belonging to the class Tremellomycetes, order Filobasidiales and genus Cryptococcus. Interestingly, 46.9% of the remaining fungi could not be assigned at the genus level indicating they are likely undescribed species.

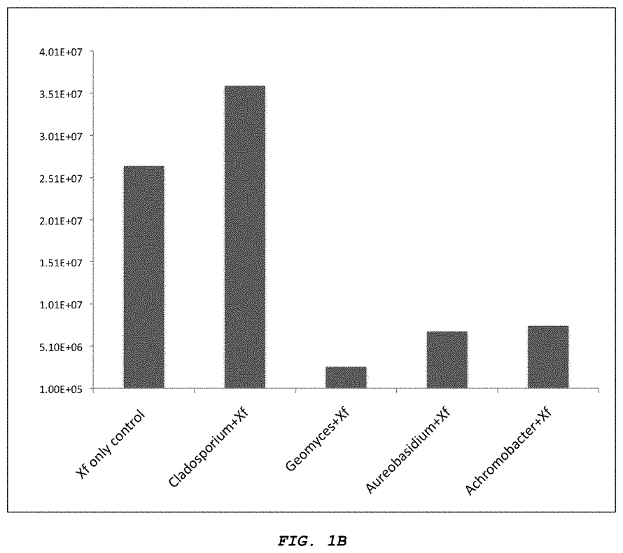

In comparison, the bacterial dataset was more robust following rarefaction method allowing the use of 72 of the 73 samples. The results showed that the bacterial communities (FIG. 2) were predominately comprised of organisms belonging to the phylum Proteobacteria (99.5%) and especially two classes, Gammaproteobacteria (89.3%) and Betaproteobacteria (10.2%). Among the Gammaproteobacteria the two dominant orders were Pseudomonadales (86.8%) and Xanthomonadales (1.7%) and were predominantly represented by Pseudomonas and Xylella, respectively. Among the Betaproteobacteria the two dominant orders were Burkholderiales (3.8%) and Rhodocyclales (1.9%) and were predominantly represented by the genera Achromobacter and Denitratisoma, respectively. Additional phyla were also present, but at a very low abundance. For example, Bacteroidetes (0.4%) was the second most abundant phylum followed by Verrucomicrobia (0.1%).

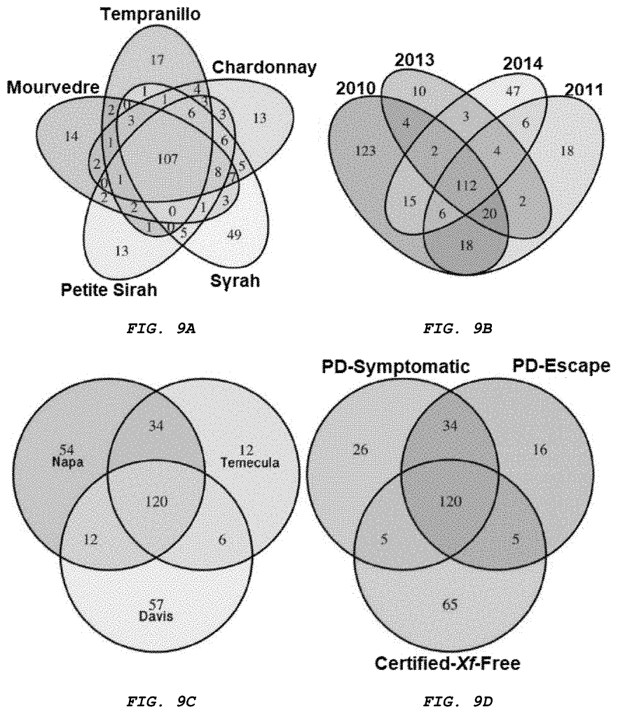

Venn diagrams were generated to assess the common and different bacterial OTUs among four variables (sampling year, vineyard location, vine condition, and grapevine cultivar) (FIG. 9A-D). OTUs had to occur in a minimum of 10% of the vines in any group to be included. Among five grape cultivars with at least 10 samples (Table 1), 107 shared OTUs were identified. Syrah had the most unique OTUs (n=47) followed by Chardonnay (n=38), Tempranillo (n=14), Mourvedre (n=13), and Petite Sirah (n=11). Among the 72 samples collected from all four years, 112 OTUs were shared between sampling dates. The year 2010 had the most unique OTUs (n=123), followed by 2014 (n=47), 2011 (n=18), and 2013 (n=10).

In addition, 120 OTUs were shared between sampling locations. FPS samples from Davis had 57 unique OTUs, Napa had 54 and Temecula had 12 unique OTUS. Similarly, 120 OTUs were shared between vine conditions with 65, 16, and 26 unique OTUs for the certified X. fastidiosa-free plants, escape, and symptomatic grapevines, respectively. Ninety-eight OTUs were shared among all the inner circles of the four Venn diagrams (condition=154; year=112, location=120; variety=107).

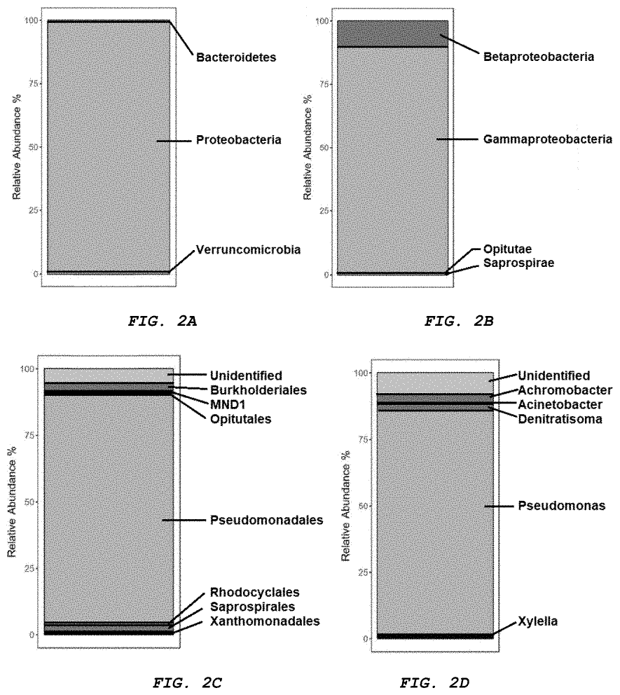

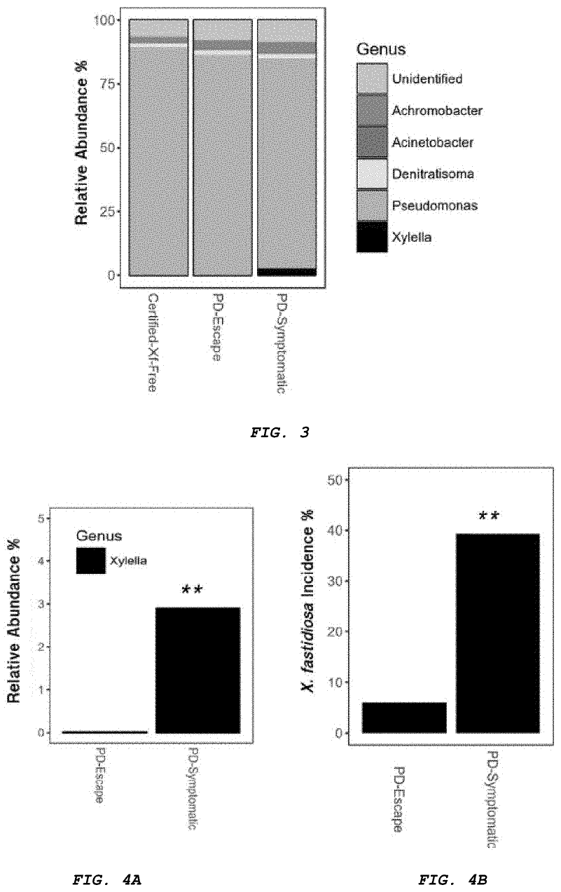

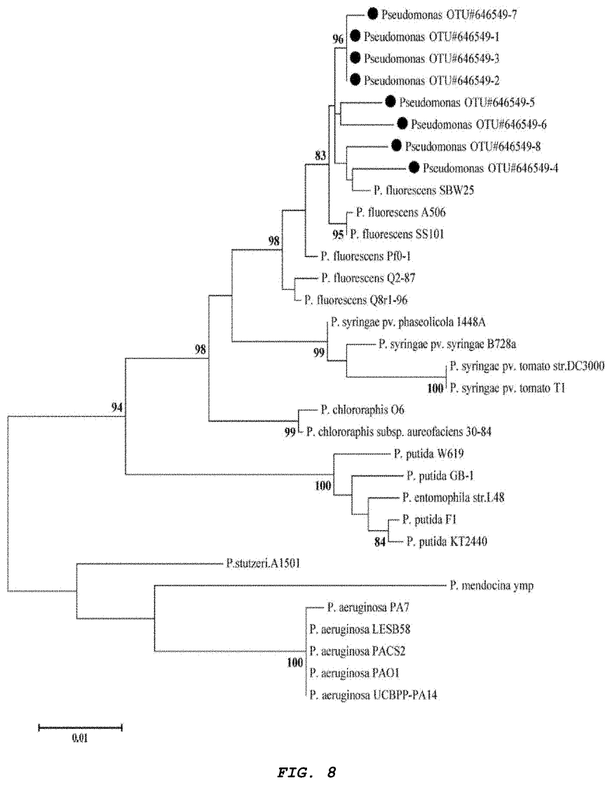

Bacterial community composition was impacted by the condition of the vine (FIG. 3). Statistical analyses showed that X. fastidiosa was found both in significantly higher abundance (P<0.01) (FIG. 4A) and at a significantly higher incidence (P<0.01) (FIG. 4B) in PD-symptomatic than PD-escape grapevines. Symptom severity on the scale of a whole plant directly correlates with an increase in Xylella titer and the reason that X. fastidiosa incidence is not reported at 100% in symptomatic vines is likely due to the heterogeneous distribution of X. fastidiosa within the xylem architecture of grapes (Clifford et al. 2013). In addition, four bacterial taxa with very low abundance correlated positively with Xylella including Bacillus, Pediococcus, Caulobacter, and Dialister (Table 2). Most notably, Pseudomonas and Achromobacter, two of the predominant phylotypes in the cane endosphere (FIG. 2), showed significant negative correlations with Xylella (Table 2). Because of the potential application of those bacteria as biological control agents for management of PD, these taxa were further identified to the species level. Chromosomal walking was used to obtain a greater portion of Pseudomonas and Achromobacter 16S rRNA gene sequence, along with their rRNA ITS sequence, producing sequences that were approximately 1,500 bp in length. From this it was determined that the species designations for these taxa were P. fluorescens or P. chlororaphis and A. xylosoxidans. A species could not be assign with confidence to the Pseudomonas OTU #646549 (i.e., the most abundant Pseudomonas OTU) using nucleotide BLAST search, with a maximum likelihood analysis to resolve species identity (FIG. 8). Results showed that the Pseudomonas sequences clustered with P. fluorescens with strong bootstrap support (98%).

TABLE 2
Bacterial operational taxonomic unites (OTUs) correlating
negatively and positively with Xylella.

| | | | | | Rel. |
|---|---|---|---|---|---|
| | | FDR | | Number of | Abundance |
| OTUs | P | corrected | r^{a} | OTUs^{b} | (%) |
| Pseudomonas | 0.000 | 0.00 | −0.84 | 84 | 82.2 |
| Achromobacter | 0.043 | 0.043 | −0.25 | 12 | 3.89 |
| Bacillus | 0.009 | 0.013 | 0.32 | 9 | 0.01 |
| Caulobacter | 0.043 | 0.043 | 0.37 | 1 | 1e−03 |
| Pediococcus | 2.8E−5 | 5.6E−5 | 0.49 | 1 | 5e−04 |
| Dialister | 0.000 | 0.000 | 0.81 | 1 | 2e−04 |

a) Pearson correlation analyses were performed between the numbers of Xylella fastidiosa sequencing reads and the relative abundance of other bacterial taxa. Standard (P) and false discovery rate (FDR) corrected probability values are presented along with the correlation coefficient (r). N=67. b) Total number of OTUs with the same genus and significant P value.

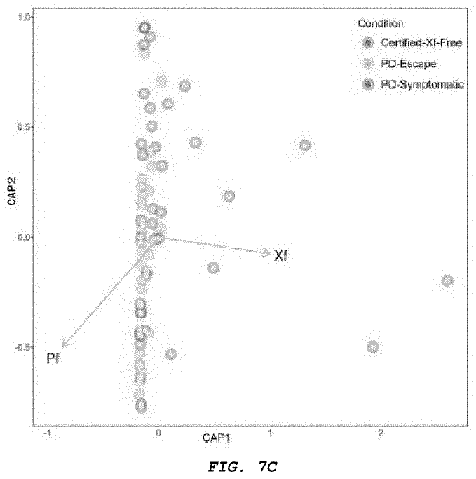

Beta-diversity plots using a Hellinger distance matrix showed evidence of compositional divergence when looking at abundance of Xylella and Pseudomonas counts (FIG. 7A-B). The clustering of bacterial communities appeared to be primarily driven by the abundance of Xylella and Pseudomonas. Adonis test revealed a significant P value and correlation coefficient for both Xylella (P<0.001, R²=0.34) and Pseudomonas (P<0.001, R²=0.28) beta-diversity metrics. A canonical correspondence of principal coordinate analysis was run to better identify the factors contributing to the variance in the dataset (FIG. 7C). Results reiterate that abundance of Xylella and abundance of Pseudomonas carry the heaviest weight on the data set with these two forces driving the data in divergent directions (P<0.001). The data points in FIG. 7C are colored by condition but the data were analyzed by X. fastidiosa abundance. Beta-diversity using Hellinger distance matrix did not show any compositional divergence belonging to any other metadata variable (i.e., condition of vine, sampling time, and vineyard location).

### Example 2

One-year-old canes from grapevines varieties Chardonnay and Cabernet Sauvignon were sampled. Although apparently healthy, these grapevines were submitted to the constant disease pressure present in Riverside County. Canes were pressure-bombed and 100 μl of the sap was plated on general fungal medium, Potato Dextrose Agar (PDA), amended with tetracycline to inhibit bacterial growth. After 2 weeks of growth at room temperature, the fungi growing were transferred to fresh PDA medium in order to obtain pure cultures. Fungal DNA was extracted from these pure cultures with a Qiagen DNA extraction kit. Following this, the ribosomal DNA was PCR-amplified (600 base pairs) and sequenced (forward and reverse). Fungal taxa were identified after comparing the r-DNA sequence to homologous sequences posted in the GenBank database. Taxa were identified from the sap of these vines, and included Aureobasidium, Cladosporium, Cryptococcus, Cochliobolus and Chaetomium.

Samples were also obtained from four varieties in two vineyards in Napa County and one vineyard in Riverside. Grapevine varieties included Chardonnay, Merlot, Riesling and Cabernet Sauvignon. One-year-old canes were collected including the wood spur from blocks that had both diseased and PD-escaped grapevines. Samples were pressure-bombed to extract the xylem sap. Following extraction, 100 μl of the xylem sap was plated on general fungal medium, Potato Dextrose Agar (PDA), amended with tetracycline to inhibit bacterial growth. In addition, wood chips were excised from the one-year-old cane and spur and were also plated on PDA-tetracycline medium. Fungi were cultured and identified as described above. The list of endophytic fungi present in escaped and diseased grapevines is presented in Table 1. Cladosporium and Aureobasidium were present in all grapevine varieties and were also found in the xylem sap.

TABLE 3
Identification and percent recovery of fungal taxa from
PD-escaped and PD-infected grapevines. Results are based
on sampling from 3 vineyards in Napa and Riverside County,
and include 4 grapevine varieties (Merlot, Cabernet Sauvignon,
Chardonnay, Riesling). Fungi were isolated from xylem sap
and one-year-old cane and spur wood.

| | Percent Recovery | |
|---|---|---|
| | Escaped Grapevines | Diseased Grapevines |
| Fungal Taxa | (n = 37) | (n = 30) |
| Cladosporium sp. | 63 | 57 |
| Aureobasidium sp. | 59 | 60 |
| Alternaria sp. | 11 | 30 |
| Cryptococcus sp. | 14 | 7 |
| Penicillium sp. | 3 | 3 |
| Geomyces sp | 3 | 7 |
| Biscogniauxia sp. | 3 | 3 |
| Nigrospora sp. | 3 | 3 |
| Peyronellae sp. | 5 | |
| Drechslera sp. | 3 | |
| Discostroma sp. | 3 | |
| Cochliobolus sp. | 3 | |
| Chaetomium sp. | 5 | |
| Aspergillus sp. | 3 | |
| Phaeosphaeria sp. | 3 | |
| Pyronema sp. | 3 | |
| Oidiodendron sp. | 3 | |
| Diplodia sp. | 3 | |
| Neofusicoccum sp. | 3 | |
| Epicoccum sp. | | 7 |
| Phomopsis sp. | | 3 |
| Fusarium sp. | | 7 |
| Cryptosporiopsis sp. | | 3 |
| Ulocladium sp. | | 13 |
| Pezizomycete sp. | | 7 |
| Didymella sp. | | 3 |

Culturable fungal candidates were evaluated in an in vitro inhibition assay for antagonism against Xf. In brief, Xf liquid cultures were adjusted to OD₆₀₀ nm=0.1 (approx. 107 CFU/ml). 300 μl of the Xf cell suspension was added to 3 ml of PD3 medium containing 0.8% agar and briefly vortexed. This mixture was overlayed onto a petri plate containing PD3 medium. A #4 size cork borer was flame sterilized and used to cut out a circle of agar containing fungal mycelium from a petri plate containing a fungal culture. This circle was placed onto the plates previously inoculated with Xf. Plates were incubated at 28° C. for 10 days and then observed for an inhibition zone around the fungal colony. Measurements were taken of the inhibition zone and recorded. Fungal species with inhibition zones were considered inhibitory to Xf.

Crude extracts of the inhibitory fungi were prepared as follows. Agar plugs of 0.5 cm diameter of each fungus were used to inoculate 250 mL liquid media, and the fungi cultivated at room temperature with shaking. After 7 days, each culture was extracted with three portions of 125 mL ethyl acetate, the extracts dried over sodium sulfate, and the solvent removed in vacuo.

Xf cultures were prepared as described above. Crude extracts from the different inhibitory fungi were re-suspended in sterile ethyl acetate to a concentration of 2 mg/ml. Volumes corresponding to a total extract mass of 1 mg, 0.1 mg, and 0.01 mg were pipetted onto sterile paper discs and allowed to dry in a laminar flow hood. Once dry, the paper discs containing the crude extracts were placed onto the Xf cultures and incubated at 28° C. for 7 days. Following this, plates were observed for a halo of inhibition around the paper disc.

Grapes cuttings var. Merlot of 2 buds were vacuum infiltrated with the fungal spore suspension, planted and placed in the greenhouse. Control plants were infiltrated with sterile water only. Shoots arising from the planted cuttings were inoculated with X. fastidiosa (Temecula strain) by mechanical needle inoculation. A sub-sample of plants were not inoculated to determine if the concentration of fungal spores used are detrimental to the grape cuttings. Planted cuttings were evaluated for PD symptoms.

Two exemplary candidate organisms (Achromobacter sp. and Geomyces sp.) decreased PD symptom severity and Xf titer in greenhouse studies.

Initial studies demonstrated that crude ethyl acetate extracts of the culture supernatants of the biocontrol candidate Cochliobolus sp. possess anti-Xf activity. Using nuclear magnetic resonance spectroscopy and mass spectrometry, this inhibitory activity was traced to a single metabolite, which we identified as radicinin (a.k.a., stemphylone). Radicinin is known to be produced by several fungi, including Cochliobolus spp. and is also known to possess antimicrobial activity.

What is claimed is:

1. A method for preventing infection and/or protecting a plant from infection by Xylella sp. microorganisms comprising inoculating the plant with (i) an anti-Xf endophytic microorganism selected from the group consisting of Pseudomonas sp. and/or Achromobacter sp. and/or (ii) an extract comprising an anti-Xf agent that inhibits Xylella sp. infection.

2. The method of claim 1, wherein the Xylella sp. is Xylella fastidiosa.

3. The method of claim 1, wherein the anti-Xf endophytic microorganism is Pseudomonas fluorescens and/or Achromobacter xylosoxidans.

4. The method of claim 1, wherein the plant is a monocotyleyledonous plant.

5. The method of claim 1, wherein the plant is a dictotyledonous plant.

6. The method of claim 1, wherein the plant is selected from the group consisting of grape, oleander, oak, almond, peach, pear, citrus, coffee, maple, mulberry, elm, sycamore, and alfalfa.

7. The method of claim 1, wherein the anti-Xf endophytic microorganism is inoculated in the xylem of the plant.

8. The method of claim 1, wherein the anti-Xf agent comprises the general structural formula selected from the group consisting of:
(a) Formula Ia: 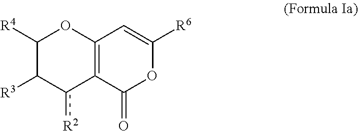
wherein,
R² is selected from the group comprising halo, CN, O, OH, NH, NH₂, S, SH, and CH₂;
R³-R⁴ are each individually selected from the group comprising H, halo, hydroxyl, cyano, thiol, amino, optionally substituted (C₁-C₆)alkyl, and optionally substituted (C₁-C₆)alkenyl; and
R⁶ is selected from the group comprising H, optionally substituted (C₁-C₆)alkyl, and optionally substituted (C₁-C₆)alkenyl, and optionally substituted heterocycle;
(b) Formula 1b: 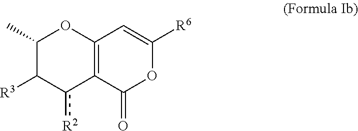
wherein,
R² is selected from the group comprising halo, CN, O, OH, NH, NH₂, S, SH, and CH₂;
R³ is individually selected from the group comprising H, halo, hydroxyl, cyano, thiol, amino, optionally substituted (C₁-C₆)alkyl, and optionally substituted (C₁-C₆)alkenyl; and
R⁶ is selected from the group comprising H, optionally substituted (C₁-C₆)alkyl, and optionally substituted (C₁-C₆)alkenyl, and optionally substituted heterocycle;
(c)

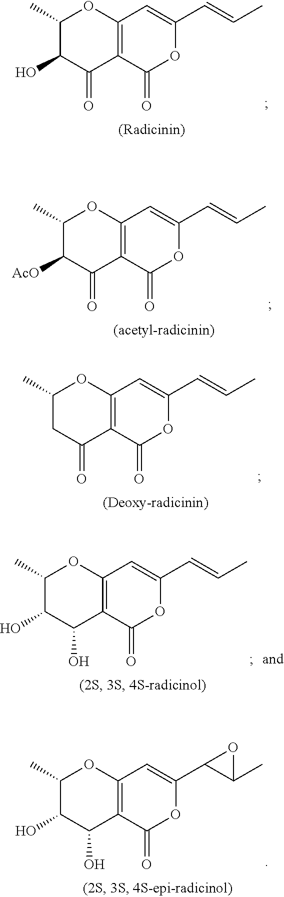

9. The method of claim 8, wherein the anti-Xf agent is administered by a syringe into the xylem.

10. A method of preparing an environment for growth of a plant susceptible to Xylella infection comprising (i) inoculating Pseudomonas sp. and/or Achromobacter sp. onto or into the soil or water, and/or (ii) inoculating an extract comprising an anti-Xf agent that inhibits Xylella sp. infection into the soil or water; and planting the plant.

11. The method of claim 10, wherein the Xylella sp. is Xylella fastidiosa.

12. The method of claim 10, wherein the anti-Xf endophytic microorganism is Pseudomonas fluorescens and/or Achromobacter xylosoxidans.

13. The method of claim 10, wherein the plant is a monocotyleyledonous plant.

14. The method of claim 10, wherein the plant is a dictotyledonous plant.

15. The method of claim 10, wherein the plant is selected from the group consisting of grape, oleander, oak, almond, peach, pear, citrus, coffee, maple, mulberry, elm, sycamore, and alfalfa.

16. The method of claim 10, wherein the anti-Xf agent comprises the general structural formula selected from the group consisting of:
(a) Formula Ia: 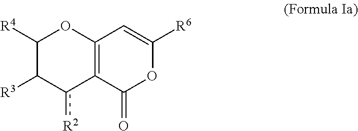
wherein,
R² is selected from the group comprising halo, CN, O, OH, NH, NH₂, S, SH, and CH₂;
R³-R⁴ are each individually selected from the group comprising H, halo, hydroxyl, cyano, thiol, amino, optionally substituted (C₁-C₆)alkyl, and optionally substituted (C₁-C₆)alkenyl; and
R⁶ is selected from the group comprising H, optionally substituted (C₁-C₆)alkyl, and optionally substituted (C₁-C₆)alkenyl, and optionally substituted heterocycle;
(b) Formula 1b: 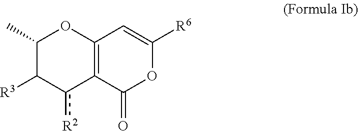
wherein,
R² is selected from the group comprising halo, CN, O, OH, NH, NH₂, S, SH, and CH₂;
R³ is individually selected from the group comprising H, halo, hydroxyl, cyano, thiol, amino, optionally substituted (C₁-C₆)alkyl, and optionally substituted (C₁-C₆)alkenyl; and
R⁶ is selected from the group comprising H, optionally substituted (C₁-C₆)alkyl, and optionally substituted (C₁-C₆)alkenyl, and optionally substituted heterocycle;
(c)

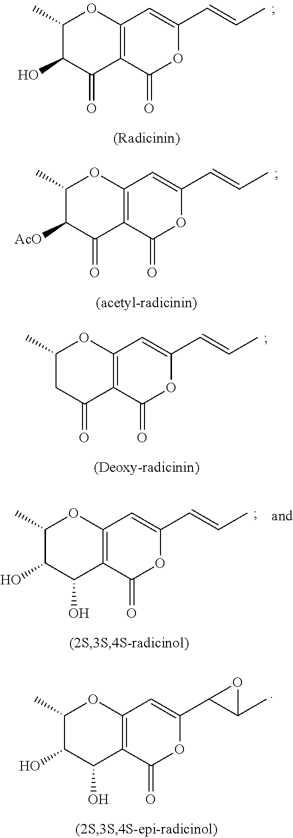

17. A method of treating a Xylella infection in a plant comprising inoculating (i) Pseudomonas sp. and/or Achromobacter sp. into the xylem of the plant, and/or (ii) inoculating an extract comprising an anti-Xf agent that inhibits Xylella sp. infection into the xylem of the plant.

18. The method of claim 17, wherein the Xylella sp. is Xylella fastidiosa.

19. The method of claim 17, wherein the anti-Xf endophytic microorganism is Pseudomonas fluorescens and/or Achromobacter xylosoxidans.

20. The method of claim 17, wherein the plant is a monocotyleyledonous plant.

21. The method of claim 17, wherein the plant is a dictotyledonous plant.

22. The method of claim 17, wherein the plant is selected from the group consisting of grape, oleander, oak, almond, peach, pear, citrus, coffee, maple, mulberry, elm, sycamore, and alfalfa.

23. The method of claim 17, wherein the anti-Xf agent comprises the general structural formula selected from the group consisting of:
(a) Formula Ia: 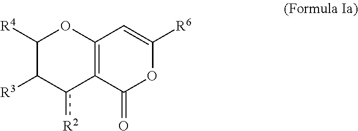
wherein,
R² is selected from the group comprising halo, CN, O, OH, NH, NH₂, S, SH, and CH₂;
R³-R⁴ are each individually selected from the group comprising H, halo, hydroxyl, cyano, thiol, amino, optionally substituted (C₁-C₆)alkyl, and optionally substituted (C₁-C₆)alkenyl; and
R⁶ is selected from the group comprising H, optionally substituted (C₁-C₆)alkyl, and optionally substituted (C₁-C₆)alkenyl, and optionally substituted heterocycle;
(b) Formula 1b: 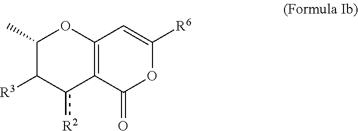
wherein,
R² is selected from the group comprising halo, CN, O, OH, NH, NH₂, S, SH, and CH₂;
R³ is individually selected from the group comprising H, halo, hydroxyl, cyano, thiol, amino, optionally substituted (C₁-C₆)alkyl, and optionally substituted (C₁-C₆)alkenyl; and
R⁶ is selected from the group comprising H, optionally substituted (C₁-C₆)alkyl, and optionally substituted (C₁-C₆)alkenyl, and optionally substituted heterocycle;
(c) [chemical structure]
